(19) United States
(12) Plant Patent Application Publication
(10) Pub. No.: US 2016/0353631 P1
(43) Pub. Date: Dec. 1, 2016

(54) Hesperozygis plant named 'HES060101'

(50) ;

(71) Applicant: USHIO SAKAZAKI, HIKONE (JP)

(72) Inventor: USHIO SAKAZAKI, HIKONE (JP)

(73) Assignee: EUROAMERICAN PROPAGATORS LLC, BONSALL, CA (US)

(21) Appl. No.: 14/545,626

(22) Filed: May 29, 2015

(51) Int. Cl. A01H 5/00

(52) U.S. Cl. PLT/263.1

(57) ABSTRACT

A new and distinct Hesperozygis plant named ‘HES060101’, characterized by its compact, upright to outwardly spreading, mounding to trailing plant habit; freely branching habit; dense and bushy appearance; early and freely flowering habit; flowers that are purple blue in color; long flowering period; and good garden performance.

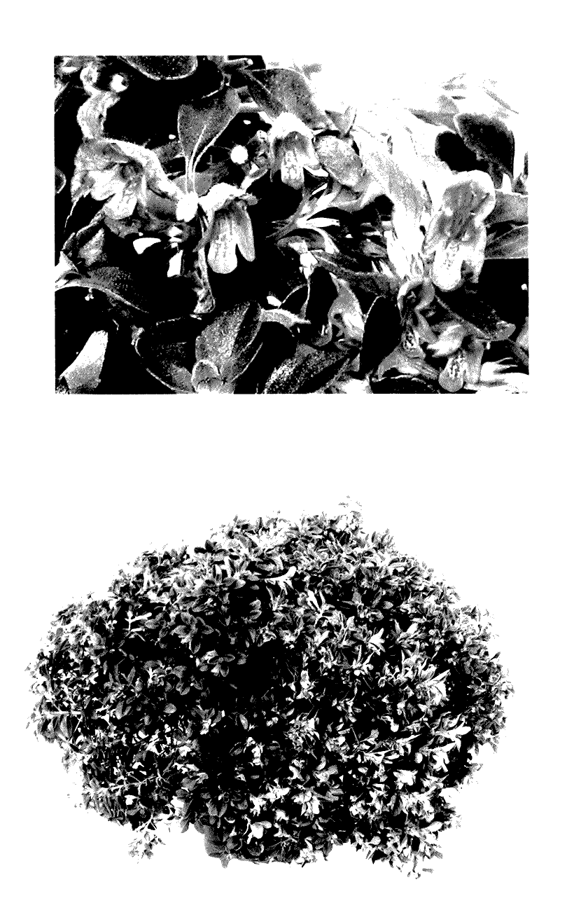

### BOTANICAL DESIGNATION

Hesperozygis hybrida

### CULTIVAR DENOMINATION

‘HES060101’

### BACKGROUND OF THE INVENTION

The present invention relates to a new and distinct cultivar of Hesperozygis plant, botanically known as Hesperozygis hybrida and hereinafter referred to by the name ‘HES060101’.

The new Hesperozygis plant is a product of a planned breeding program conducted by the Inventor in Higashiomi, Shiga, Japan. The objective of the breeding program is to create new uniform and freely-branching Hesperozygis plants with fragrance and attractive flowers.

The new Hesperozygis plant originated from a cross-pollination made by the Inventor on Apr. 19, 2005 in Higashiomi, Shiga, Japan of an unnamed proprietary selection of Hesperozygis kleinii, not patented, as the female, or seed, parent with Hesperozygis hybrida ‘Sunminbu’, disclosed in U.S. Plant Pat. No. 11,677, as the male, or pollen, parent. The new Hesperozygis plant was discovered and selected by the Inventor as a single flowering plant within the progeny of the stated cross-pollination in a controlled environment in Higashiomi, Shiga, Japan on Sep. 20, 2006.

Asexual reproduction of the new Hesperozygis plant by vegetative terminal cuttings in a controlled environment in Higashiomi, Shiga, Japan since Sep. 25, 2007 has shown that the unique features of this new Hesperozygis plant are stable and reproduced true to type in successive generations.

### SUMMARY OF THE INVENTION

Plants of the new Hesperozygis have not been observed under all possible combinations of environmental conditions and various cultural practices. The phenotype may vary somewhat with variations in environmental conditions such as temperature and light intensity without, however, any variance in genotype.

The following traits have been repeatedly observed and are determined to be the unique characteristics of ‘HES060101’. These characteristics in combination distinguish ‘HES060101’ as a new and distinct Hesperozygis plant:
1. Compact, upright to outwardly spreading and mounding plant habit.
2. Freely branching habit; dense and bushy appearance.
3. Early and freely flowering habit.
4. Flowers that are purple blue in color.
5. Long flowering period.
6. Good garden performance.

The new Hesperozygis plant can be compared to plants of the female parent selection. Plants of the new Hesperozygis differ primarily from plants of the female parent selection in flower color as plants of the female parent selection have purple-colored flowers. In addition, plants of the new Hesperozygis flower for a longer period of time than plants of the female parent selection.

The new Hesperozygis plant can be compared to plants of the male parent, ‘Sunminbu’. Plants of the new Hesperozygis differ primarily from plants of ‘Sunminbu’ in the following characteristics:
1. Plants of the new Hesperozygis are more vigorous than plants of ‘Sunminbu’.
2. Plants of the new Hesperozygis have narrower leaves than plants of ‘Sunminbu’.
3. Plants of the new Hesperozygis flower earlier and for a longer period of time than plants of ‘Sunminbu’.
4. Plants of the new Hesperozygis and ‘Sunminbu’ differ in flower color as plants of ‘Sunminbu’ have lavender blue-colored flowers.

Plants of the new Hesperozygis can be compared to plants of Saturozygis ‘Usmint2’, disclosed in U.S. Plant Pat. No. 16,790. In side-by-side comparisons plants of the new Hesperozygis differed primarily from plants of ‘Usmint2’ in flower size and color as plants of ‘Usmint2’ had longer pink-colored flowers that did not change in color with development. In addition, plants of the new Hesperozygis flowered later than plants of ‘Usmint2’.

### BRIEF DESCRIPTION OF THE PHOTOGRAPHS

The accompanying colored photographs illustrate the overall appearance of the new Hesperozygis plant showing the colors as true as it is reasonably possible to obtain in colored reproductions of this type. Colors in the photographs may differ slightly from the color values cited in the detailed botanical description which accurately describe the colors of the new Hesperozygis plant. The photograph at the bottom of the sheet comprises a side perspective view of a typical flowering plant of ‘HES060101’ grown in a container. The photograph at the top of the sheet is a close-up view of a typical flowering plant of ‘HES060101’.

### DETAILED BOTANICAL DESCRIPTION

The aforementioned photographs and following observations, measurements and values describe plants grown during the winter and early spring in 16.5-cm containers in a coldframe greenhouse in Bonsall, Calif. During the production of the plants, day temperatures ranged from 10° C. to 35° C. and night temperatures ranged from 7° C. to 21° C. Plants were 24 weeks old when the photographs and description were taken. In the following description, color references are made to The Royal Horticultural Society Colour Chart, 2007 Edition, except where general terms of ordinary dictionary significance are used.
Botanical classification: Hesperozygis hybrida ‘HES060101’.
Parentage:
   Female, or seed, parent.—Unnamed proprietary selection of Hesperozygis kleinii, not patented.
   Male, or pollen, parent.—Hesperozygis hybrida ‘Sunminbu’, disclosed in U.S. Plant Pat. No. 11,677.
Propagation:
   Type.—By vegetative terminal cuttings.
   Time to initiate roots, summer.—About three weeks at temperatures ranging from 24° C. to 27° C.
   Time to initiate roots, winter.—About four weeks at temperatures ranging from 21° C. to 24° C.
   Time to produce a rooted plant, summer.—About six weeks at temperatures ranging from 24° C. to 27° C.
   Time to produce a rooted plant, winter.—About seven weeks at temperatures ranging from 18° C. to 21° C.
   Root description.—Fine, fibrous; white in color.
   Rooting habit.—Freely branching; medium density.
Plant description:
   Plant and growth habit.—Compact, upright to outwardly spreading and mounding plant habit; nearly spherical in shape; freely branching habit with about ten primary lateral branches each with multiple secondary branches; pinching enhances branching; dense and bushy appearance; vigorous growth habit.
   Plant height.—About 17.5 cm.
   Plant diameter.—About 42 cm.
Lateral branch description:
   Length.—Highly variable, about 28 cm.
   Diameter.—About 5 mm.
   Internode length.—About 1.6 cm.
   Strength.—Moderately strong to strong.
   Aspect.—Initially upright then outwardly spreading.
   Texture.—Pubescent, minute.
   Color, developing.—Close to 146C.
   Color, developed.—Close to N199A.
Leaf description:
   Arrangement.—Opposite; leaves simple.
   Fragrance.—Strongly fragrant, intense; spicy.
   Length.—About 2 cm.
   Width.—About 1.2 cm.
   Shape.—Elliptical.
   Apex.—Broadly acute to rounded.
   Base.—Attenuate.
   Margin.—Mostly entire with shallow crenate-like notches.
   Texture, upper and lower surfaces.—Sparsely pubescent, minute.
   Venation pattern.—Pinnate; arcuate.
   Color.—Developing and fully expanded leaves, upper surface: Close to N137A; venation, close to 147B. Developing and fully expanded leaves, lower surface: Close to 147B; venation, close to 146C.
   Petioles.—Length: About 1 cm. Diameter: About 1 mm. Texture, upper and lower surfaces: Pubescent, minute. Color, upper and lower surfaces: Close to 146C.
Flower description:
   Flower type and flowering habit.—Single salverform flowers arising from leaf axils; freely flowering habit; typically in clusters with about three per leaf axil, about six per node and more than 1,250 flowers developing per plant; flowers face upright to leaning outwardly.
   Fragrance.—None detected.
   Natural flowering season.—Early flowering habit, plants of the new Hesperozygis begin flowering about ten weeks after planting; plants flower continuously from spring though the summer until autumn in Southern California.
   Flower longevity.—Individual flowers last about four or five days on the plant; flowers not persistent.
   Flower buds.—Length: About 1.3 cm. Diameter: About 3 mm. Shape: Oblong. Texture: Pubescent, minute. Color: Close to 79D.
   Flower diameter.—About 9 mm.
   Flower length.—About 2.4 cm.
   Flower throat diameter.—About 4 mm by 6 mm.
   Flower tube length.—About 2.4 cm.
   Flower tube diameter, base.—About 2 mm.
   Corolla.—Arrangement: Five fused petals opening into a flared tube. Petal lobe length (from throat): About 2 mm to 4 mm. Petal lobe width: About 2 mm to 6 mm. Petal shape: Oval. Petal apex: Rounded. Petal margin: Entire. Petal texture, upper surface: Smooth, glabrous. Petal texture, lower surface: Pubescent, minute. Throat texture: Smooth, glabrous. Tube texture: Pubescent, minute.
   Color.—Petal lobe, when opening, upper surface: Close to 84A. Petal lobe, when opening, lower surface: Close to N87D. Petal lobe, fully opened, upper and lower surfaces: Close to 84A; with development, color becomes closer to N82D. Throat: Close to 84A with irregular splotches (nectar guides) close to 83B; with development, color becomes closer to N82D. Tube: Close to 84A; with development, color becomes closer to N82D.
   Calyx.—Arrangement: Tubular calyx with five sepals fused towards the base and arranged in a single whorl. Sepal length: About 7 mm. Sepal width: About 1 mm. Sepal shape: Lanceolate. Sepal apex: Acuminate. Sepal margin: Entire. Sepal texture, upper surface: Smooth, glabrous; towards the apex, pubescent, minute. Sepal texture, lower surface: Pubescent, minute. Sepal color, upper surface: Close to 146D. Sepal color, lower surface: Close to 146C.
   Peduncles.—Length: About 2 mm. Diameter: About 1 mm. Angle: About 30° to 45° from stem axis. Strength: Moderately strong. Texture: Pubescent, minute. Color: Close to 146B.
   Reproductive organs.—Stamens: Quantity: Two per flower; adnate to the throat. Filament length: About 1 cm. Filament color: Close to 84B. Anther length: About 1 mm. Anther shape: Lanceolate. Anther color: Close to 83D. Pollen amount: Scarce. Pollen color: Close to NN155A. Pistils: Quantity: One per flower. Pistil length: About 2.1 cm. Style length: About 2 cm. Style color: Close to 84B to 84C. Stigma shape: Two-parted and curved. Stigma color: Close to 84A. Ovary color: Close to 145B. Seeds and fruits: Seed and fruit development have not been observed on plants of the new Hesperozygis.
Garden performance: Plants of the new Hesperozygis have been observed to have good garden performance and tolerate wind, rain and temperatures ranging from about 1° C. to about 40° C.
Pathogen & pest resistance: Plants of the new Hesperozygis have not been observed to be resistant to pathogens and pests common to Hesperozygis plants.

It is claimed:

1. A new and distinct Hesperozygis plant named ‘HES060101’ as illustrated and described.
